(19) United States
(12) Patent Application Publication
(10) Pub. No.: US 2025/0191492 A1
(43) Pub. Date: Jun. 12, 2025

(54) Virtual Reality System and Method for Enhanced Medical Training with Advanced Hand Tracking

(71) Applicant: Ngo Kiu Cheung, Hong Kong (CN)

(72) Inventor: Ngo Kiu Cheung, Hong Kong (CN)

(21) Appl. No.: 18/533,258

(22) Filed: Dec. 8, 2023

(51) Int. Cl. G09B 19/00; A61B 34/10; G06F 3/01

(52) U.S. Cl. CPC G09B 19/00; A61B 34/10; G06F 3/014; G06F 3/016; G06F 3/017; A61B 2034/105

(57) ABSTRACT

The present invention relates to a virtual reality (VR) system and method for enhancing medical training. Integrating advanced hand tracking technology with a VR headset, this system offers an immersive training environment for a variety of medical procedures, including CPR, emergency medical interventions, and physical examinations. The hand tracking sensors provide precise monitoring of hand movements, allowing for gesture-based interaction without physical controllers. The system includes several modules for different medical scenarios, each designed to accurately replicate medical techniques and provide real-time feedback. Key innovations include haptic feedback, adjustable difficulty levels, interactive tutorials, and an AI-driven virtual instructor for a personalized training experience. The system is particularly beneficial for training healthcare professionals in critical skills necessary for effective patient care in emergency situations, representing a significant improvement over traditional training methods.

### FIELD OF INVENTION

The invention relates to medical training technologies, particularly the use of virtual reality (VR) for simulating medical procedures. It focuses on enhancing realism and interactivity in medical training through advanced hand tracking technology integrated with VR environments.

### BACKGROUND

The development of virtual reality (VR) technology has revolutionized various fields, including medical training. Traditional methods of medical training, particularly for procedures like Cardiopulmonary Resuscitation (CPR) and other emergency medical procedures, have relied heavily on manikin-based practice and in-person instruction. These methods, while effective, present several limitations. The physical presence of both the trainee and instructor is essential, and the training experiences may not accurately represent the diversity of real-life medical scenarios. Additionally, the cost and logistics of organizing such training sessions can be prohibitive.

In recent years, VR simulations have emerged as a promising alternative, offering immersive and interactive environments for medical training. However, most existing VR medical training applications are limited by their reliance on handheld controllers. While these controllers facilitate interaction within the virtual environment, they often fail to mimic the complex and nuanced movements required in medical procedures. This limitation undermines the realism and effectiveness of the training, as the use of controllers cannot replicate the tactile feedback and precise motor skills required in real medical scenarios.

The advent of advanced hand tracking technology presents a unique opportunity to enhance VR medical training. Unlike traditional controllers, hand tracking technology enables the detection and analysis of intricate hand movements and gestures. This advancement is crucial in medical training, where the precision of hand movements can be critical, such as in performing chest compressions in CPR or using specific medical instruments. The ability to track each finger and joint allows for a more accurate and detailed replication of real-life medical procedures within the VR environment.

Moreover, the incorporation of hand tracking in VR medical training aligns with the growing demand for more realistic, cost-effective, and accessible training tools. By simulating various medical scenarios, including emergency situations like cardiac arrest, VR simulations with hand tracking can provide a more comprehensive and engaging learning experience. They offer the potential to improve upon traditional CPR methods, which are crucial for effective patient care in emergencies.

Despite the advancements in VR and hand tracking technology, their integration in medical training is not widespread. The limitations of existing VR medical simulations, mainly their reliance on controllers, highlight the need for a more immersive and realistic training tool. This necessity is particularly pronounced in the context of medical emergencies, where the ability to perform procedures accurately and confidently can significantly impact patient outcomes. The integration of hand tracking technology in VR medical training is thus a timely and crucial development, addressing the current shortcomings in medical training methodologies and paving the way for more effective and realistic training solutions.

It is within this context that the present invention is provided.

### SUMMARY

This invention relates to a system and method for implementing virtual reality (VR) simulations to enhance medical training. The core of the invention lies in the integration of advanced hand tracking technology with VR simulations, creating a comprehensive and immersive training environment without the need for physical controllers. The system offers a novel approach to medical training by allowing for gesture-based interaction with simulated medical objects and scenarios, including a range of procedures such as CPR, emergency delivery, and physical examinations.

The invention comprises a VR headset equipped with hand tracking sensors, enabling the detection and analysis of intricate hand movements and gestures. This technology allows for a highly realistic training experience, closely mimicking the tactile and motor skills required in actual medical procedures. The system provides real-time feedback, guiding users through correct procedure steps and techniques.

Key features include modules for various medical scenarios like chest compression, rescue breath with bag-valve-mask, defibrillation, and medication injection, each designed to ensure accuracy and fidelity in replicating medical techniques. The system also offers adjustable difficulty levels, interactive tutorials, and an AI-driven virtual instructor for personalized training experiences.

An optional addition to the system is the incorporation of a connected device such as a haptic glove type device to provide haptic feedback, simulating the tactile sensations associated with different medical procedures. Additionally, the system may include a performance tracking and analysis module, offering detailed feedback and improvement recommendations post-simulation.

The invention significantly improves upon traditional medical training methods, offering a cost-effective, accessible, and realistic training tool. Its potential to enhance the quality of medical training and, consequently, patient care, especially in emergency situations, is substantial.

### BRIEF DESCRIPTION OF THE DRAWINGS

Various embodiments of the invention are disclosed in the following detailed description and accompanying drawings.

FIG. 1 is a flowchart that illustrates the step-by-step process within a virtual reality (VR) medical training system, depicting the sequence of operational steps from initiation to conclusion of a medical simulation.

FIG. 2A is a first-person perspective within a VR medical training scenario, showing the user's hands interacting with a virtual patient, emphasizing the detailed hand tracking technology and the simulated medical environment.

FIG. 2B is a continuation from FIG. 2A, this figure displays the user's hands utilizing a virtual oxygen supply tool on the virtual patient, demonstrating the system's capability to simulate and respond to the medical procedure of artificial ventilation.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention.

The embodiments of the invention described herein are exemplary and numerous modifications, variations, and alterations could be made without departing from the scope of the invention as set forth in the claims. For instance, while the system is described primarily for CPR and emergency medical procedures, it can be adapted for a wide range of medical training scenarios. The specifics of the hand tracking technology, VR headset design, and feedback mechanisms may also vary. These and other modifications are intended to be encompassed within the scope of the invention.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

“Virtual Reality (VR)”: A simulated environment created with software and experienced through sensory stimuli provided by a computer system and VR headset.

“Hand Tracking Technology”: Refers to technology used to detect and interpret the movements and positions of a user's hands and fingers.

“Medical Training Scenario”: A simulated medical procedure or situation within the VR environment designed for the training of medical professionals or students.

### DESCRIPTION OF DRAWINGS

The following detailed description is intended to convey a thorough understanding of the embodiments described herein and is not intended to limit the scope of the invention to those embodiments alone. Instead, the detailed description will describe the functionality and construction of the system and method for implementing virtual reality (VR) simulations in a medical training context, which incorporates advanced hand tracking technology to facilitate an immersive, interactive educational experience.

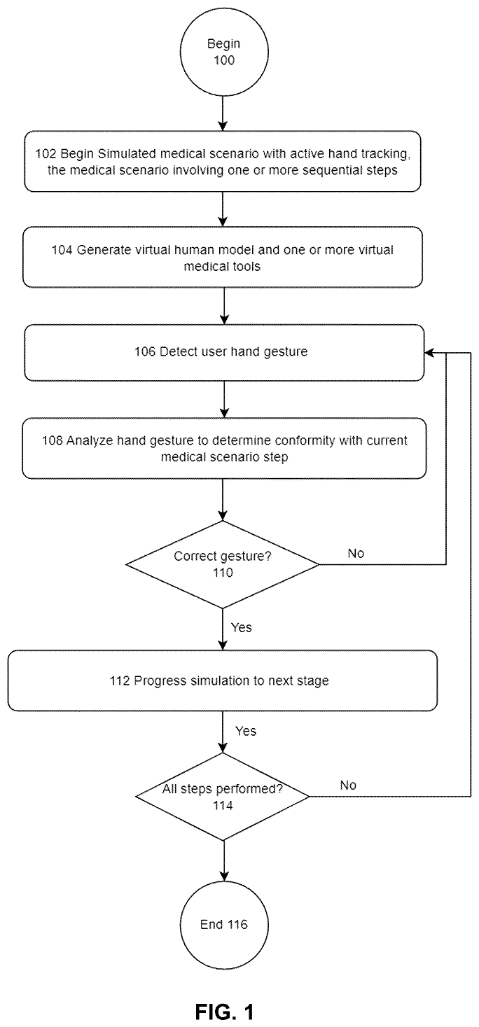

Thus, referring to FIG. 1, a flowchart is provided illustrating the operational steps of the disclosed virtual reality (VR) medical training system, which incorporates advanced hand tracking technology to simulate medical scenarios for training purposes. The process initiates at step 100, where the user begins the VR simulation.

At step 102, the system launches a simulated medical scenario, which may range from basic medical procedures to complex surgical operations. This scenario is equipped with active hand tracking, allowing for the accurate monitoring of the user's hand movements as they navigate through the sequential steps of the medical process. The scenarios are designed to provide a comprehensive and immersive learning experience, comparable to real-life medical situations but without the need for physical controllers.

In step 104, the system generates a highly detailed virtual human model, alongside a suite of virtual medical tools pertinent to the scenario at hand. For instance, if the simulation involves a CPR procedure, the virtual environment will include a patient requiring chest compressions and potentially a defibrillator or other resuscitation equipment.

Following this, step 106 involves the detection of the user's hand gestures. The integrated hand tracking sensors within the VR headset capture the user's movements, identifying gestures that correspond to medical tasks such as chest compressions, the use of a stethoscope, or the handling of surgical instruments.

Step 108 is crucial as it involves analyzing the detected hand gestures against the procedural steps required in the medical scenario. The system's software module compares the user's gestures to the correct execution of the medical procedure. This real-time analysis allows the system to provide immediate feedback on the user's performance, ensuring that each action is carried out with precision.

If the hand gesture is determined to be correct at step 110, reflecting the user's successful completion of a task such as selecting the appropriate tool for an incision, the simulation progresses to the next stage at step 112. This progression is a testament to the user's understanding and correct application of the medical procedure.

The decision point at step 114 ascertains whether all procedural steps have been completed. If further actions are required, the simulation returns to step 106, continuing the interactive training. Upon completion of all tasks, the process concludes at step 116.

The method provides the capability to guide the user through various medical procedures, from CPR to emergency deliveries, leveraging the hand tracking sensors to provide a realistic training environment. This system not only improves upon the conventional CPR training methods, which typically involve manikins, but it also offers a cost-effective and accessible solution for medical education, enabling users to develop the muscle memory and tactile skills necessary for patient care.

The system's functionality is further enhanced by the sophisticated virtual patient model that dynamically interacts with users, offering various medical scenarios, including cardiac arrest and post-resuscitation conditions. The system's optional ability to integrate with a haptic glove device to provide haptic feedback may add another layer of realism, simulating the tactile sensations associated with medical procedures such as chest compressions.

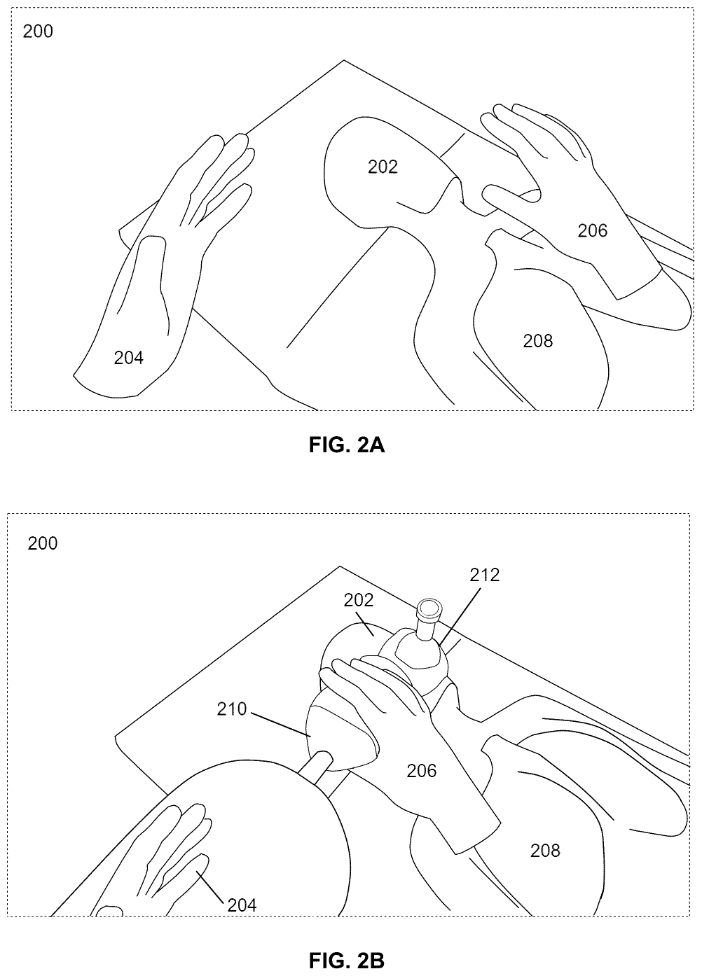

FIG. 2A illustrates a first-person view from a virtual reality (VR) display 200 as experienced by a user during a medical training simulation. The user's left hand 204 and right hand 206 are depicted within the VR environment, demonstrating the system's hand tracking capabilities. The hands are shown interacting with a virtual patient model 202, which is lying on a simulated bed within the virtual environment. Notably, the patient model's lungs are accentuated, possibly indicating a focal point for the medical procedure being trained, such as in the administration of CPR or a respiratory examination.

The VR display 200 provides the user with an immersive visual representation of the medical scenario, including the patient model and the environment in which the user operates. The virtual patient model 202 serves as the central element of the simulation, offering a realistic platform for the user to perform medical procedures. The hand tracking technology employed by the system accurately captures the position, orientation, and gestures of the user's hands in real-time, as illustrated by the natural positioning of the hands in relation to the patient model.

The left hand 204 and the right hand 206 of the user are represented in such a way that mirrors the user's physical movements, allowing for a seamless translation of real-world actions into the virtual simulation. This enables the user to practice procedures with a high degree of precision, as required in actual medical settings. The highlighted lungs on the patient model suggest that the system provides visual cues to guide the user through the procedure, emphasizing critical areas of interaction.

FIG. 2B is a continuation of the immersive virtual reality (VR) simulation for medical training. Here, the user's interaction progresses to the utilization of a virtual oxygen supply tool, comprising a pump 210 and a mask 212, within the VR medical scenario.

The user's left hand 204 and right hand 206 are shown in the act of placing the virtual oxygen mask 212 over the mouth of the virtual patient model 202. The right hand 206 is depicted squeezing the virtual pump 210. This action within the VR simulation corresponds to the delivery of artificial ventilation, a critical component of life-saving procedures such as Cardiopulmonary Resuscitation (CPR).

As the user operates the pump 210, the virtual lungs 208 of the patient model are shown to expand. This visual cue within the simulation is indicative of the VR system's dynamic response to user input, reflecting the simulation's fidelity in imitating the physiological response to resuscitation efforts. It also serves as real-time feedback to the user, ensuring that the necessary medical procedure is performed correctly and effectively.

The accurate representation of the user's hands and the responsive nature of the virtual tools and patient model in FIG. 2B exemplify the system's advanced hand tracking capabilities. These capabilities enable precise simulation of medical interventions, allowing medical professionals to practice and refine their techniques in a controlled, risk-free environment.

By providing such interactive tools as the oxygen supply pump and mask, the VR system not only enhances the realism of the medical training but also provides a platform for users to learn and practice the coordination and manual dexterity required in emergency medical situations.

### Hardware Components

A virtual reality (VR) system as described herein can be any suitable type of computing device capable of delivering immersive interactive experiences. A computing device in the context of VR may be a uniprocessor or multiprocessor machine, equipped to handle complex simulations with high computational demands. Accordingly, such a VR system may include one or more processors specifically designed or adapted for VR applications. Examples of suitable processors include those commonly found in gaming consoles, high-performance PCs, or dedicated VR hardware, such as microprocessors, graphics processing units (GPUs), central processing units (CPUs), digital signal processors (DSPs), and systems on a chip (SoC) optimized for rendering 3D environments and processing real-time user interactions within these environments.

Additionally, the VR system may include one or more memories, which are crucial for storing the intricate simulations and user interaction data required for a comprehensive VR experience. The memory storage may consist of various types, including volatile and non-volatile memory, such as random access memory (RAM), solid-state drives (SSD), and other forms of digital storage capable of high-speed read/write operations to facilitate the real-time demands of VR. These memories are designed to store computer-executable instructions that, when processed by the VR system's processors, enable the intricate simulation of virtual environments and the accurate tracking of user movements within these environments.

These processors are operatively connected to the memory or memories to facilitate the rapid retrieval and processing of instructions and data. This connection is often mediated through high-speed buses or direct memory access channels, which are crucial for the low-latency responses required in VR systems.

The VR system is also equipped with various input and output devices tailored for VR applications, including but not limited to head-mounted displays (HMDs), hand tracking sensors, haptic feedback devices such as gloves, and spatial audio systems. These components work in unison to deliver a seamless and immersive VR experience, simulating the nuances of real-world interactions within a virtual space.

For networked VR experiences or cloud-based simulations, the VR system may include network communication hardware such as Ethernet cards, Wi-Fi adapters, or other wireless communication modules that enable the VR system to connect to local networks or the internet.

The control logic of the VR system, implemented via software, firmware, or dedicated hardware modules, is configured to execute a set of operations that translate user actions into interactions within the virtual environment. This logic is responsible for the real-time interpretation of hand gestures and movements, translating them into meaningful interactions with virtual objects or scenarios, as prescribed by the VR simulation software.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the method and system have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A computer-implemented method for conducting medical training using virtual reality (VR), the method comprising:
providing a VR environment through a VR headset equipped with hand tracking sensors, wherein the VR environment includes a virtual human model simulating a patient in a medical scenario;
detecting, via the hand tracking sensors, hand gestures of a user interacting with the virtual human model, wherein the hand gestures correspond to medical procedures relevant to the simulated medical scenario;
analyzing the detected hand gestures to determine conformity with predetermined medical procedure steps, wherein the analysis includes assessing hand poses, movements, and interactions with virtual medical instruments within the VR environment;
providing real-time feedback to the user based on the analysis of the hand gestures, wherein the feedback includes visual, auditory, or haptic signals indicating the correctness of the hand gestures in performing the medical procedures; and
progressing the medical scenario within the VR environment in response to the user correctly performing a sequence of the medical procedure steps, as determined by the analysis of the hand gestures.

2. The method of claim 1, wherein the medical procedures include Cardiopulmonary Resuscitation (CPR) and the hand gestures include motions for chest compression, manual bagging, defibrillation, and medication injection.

3. The method of claim 2, wherein the chest compression module includes monitoring the rate and depth of chest compressions and providing feedback to the user based on the adherence to predetermined compression techniques.

4. The method of claim 2, wherein the rescue breath module involves the user performing an “EC” maneuver on a virtual bag-valve-mask to simulate manual ventilation.

5. The method of claim 1, wherein the medical scenario simulation includes a virtual display screen or an audio signal to confirm the successful completion of a step in the medical procedure.

6. The method of claim 1, further comprising providing guidance to the user on the sequence and method of carrying out each step of the medical procedure, including visual indicators and text labels for medical tools within the VR environment.

7. The method of claim 1, wherein the virtual human model is capable of simulating various medical conditions including cardiac arrest and post-resuscitation conditions.

8. The method of claim 1, wherein the hand tracking sensors are capable of detecting the pose and movement of each finger and joint of the user's hands.

9. The method of claim 1, wherein the VR environment is controlled by a software module on the headset processor, which interprets hand tracking data to control interactions based on the medical training simulations.

10. The method of claim 1, wherein the medical procedures include performing a physical examination using techniques such as auscultation and percussion, with the hand tracking technology evaluating the user's technique.

11. The method of claim 1, wherein the system includes a feedback module for coordinating with a haptic glove device for providing haptic feedback to the user, simulating the tactile sensation associated with the medical procedures being performed in the VR environment.

12. The method of claim 1, further comprising a module for adjusting the difficulty level of the simulations based on the user's experience or proficiency level, allowing for tailored training experiences ranging from beginner to advanced scenarios.

13. The method of claim 1, wherein the VR environment includes an interactive tutorial mode, guiding the user through each step of the medical procedure before entering the full simulation, thereby enhancing the user's understanding and preparation.

14. The method of claim 1, wherein the system incorporates an AI-driven virtual instructor that provides real-time guidance and corrections during the simulation, based on the user's performance and adherence to medical protocols.

15. The method of claim 1, further including a performance tracking and analysis module that records and analyzes the user's actions during the simulation, providing detailed feedback and recommendations for improvement post-simulation.
